(19) United States
(12) Patent Application Publication
(10) Pub. No.: US 2011/0301429 A1
(43) Pub. Date: Dec. 8, 2011

(54) METHOD FOR REMOTE DIAGNOSTIC MONITORING AND SUPPORT OF PATIENTS AND DEVICE AND TELEMEDICAL CENTER

(76) Inventor: Sascha Henke, Weil Der Stadt (DE)

(21) Appl. No.: 13/133,632

(22) PCT Filed: Oct. 30, 2009
(86) PCT No.: PCT/EP09/64353

§ 371 Date: Aug. 25, 2011

(30) Foreign Application Priority Data
Dec. 10, 2008 (DE) 10 2008 054 442.6

(51) Int. Cl. A61B 5/00

(52) U.S. Cl. 600/300

(57) ABSTRACT

Vital data are continuously recorded and/or measured for the remote diagnostic monitoring and support of patients. The vital data are interpreted and evaluated using signal technology with regard to their trend and the context in which they were recorded/measured. The vital data are linked to a patient profile and evaluated based on threshold values in order to detect deviations of the patient's condition from a previously established target condition. A categorization is made whether, based on the evaluation, an interaction with the patient is required immediately, in a predefined time frame, or not at all.

### FIELD OF THE INVENTION

The present invention relates to a method for remote diagnostic monitoring and support of patients and a device and telemedical center.

### BACKGROUND INFORMATION

In conventional telemedical systems, the vital data of patients, for example, blood pressure, weight, EKG, etc., are measured and forwarded to a so-called telemedical center. These data are evaluated manually or automatically. The patients are served by the medical staff within or outside of the telemedical center.

A health monitoring system is described in U.S. Patent Application Publication No. 2004/0117207 in which health-relevant data of a patient are collected. Based on these collected data, a health center performs evaluations for the purpose of determining whether it is necessary to change a patient's therapy program. A terminal at the patient's location is made up of a hand-held microprocessor having alphanumeric input and a display. Monitoring systems for blood sugar may be connected via a data management unit.

A monitoring system for patients is described in U.S. Pat. No. 6,248,065 which regularly retrieves health data and also enters into interaction with the patient via a monitoring program.

### SUMMARY

The features described herein, i.e., a continuous recording and/or measurement of vital data of a patient, an interpretation and evaluation of the vital data with regard to their trend and the context in which they were recorded/measured using signal technology, a linking of the vital data to a patient profile and evaluation, based on threshold values, in order to detect deviations of the patient's condition from a previously established target condition, a categorization whether, based on the evaluation, an interaction with the patient is necessary immediately, in a predefined time frame or not at all, make it possible to automate medical decisions, automate therapy recommendations, and accordingly put therapy recommendations in line with current guidelines; however, it is also possible to check for cross-reactions of medications automatically. This results in a gain in quality and efficiency. An interpretation and evaluation of the recorded vital data by signal technology, in particular based on a therapy plan, makes it possible to reduce contacts with a medical center (gain in efficiency), or additional measurements/requests for information may be initiated at the patient's location in order to make treatment in line with a therapy plan possible. Context sensitivity alone makes it possible to interpret the vital data meaningfully. The absolute value of the vital data is not a deciding factor for vital data. The decisive information is obtained from the trend and the context. Since a very large number of patients are older or multimorbid, interaction appropriate to the user is a significant added value for acceptance and is ultimately one of the deciding factors for the medical success of a telemedical application.

In a telemedical center, the vital data of the patients are linked to a patient profile and are compared using threshold values which have been established medically in order to detect deviations of the patient's condition from the intended target condition. The patients may, for example, be classified into three levels (not requiring interaction, requiring interaction, i.e., interaction in a predefined time frame, and urgently requiring interaction, i.e., immediate contact). This triage of patients may be performed automatically or manually in a medical center. The medical center initiates additional (medical) steps in order for the patient to receive medical treatment via information and recommendations or instructions.

According to an example embodiment, feedback from a telemedical center as to whether the vital data were transmitted successfully and are valid is sent to the patient. This provides the patients the certainty that their measured values have been conveyed and are within a tolerable range. Moreover, it may be reported to them in the feedback that the medical center will arrange to have someone sent to them to provide them any assistance that may be necessary.

It is advantageous to evaluate the vital data in line with a medical therapy plan in a terminal at the patient's location and additional measurements of vital data or information inputs may be initiated or requested of the patient if necessary from this evaluation.

If decision-making processes which are automated in particular with regard to the individual indications and patient are carried out in line with therapy plans in the medical center, the medical staff within the medical center receive a kind of presorting of the condition of the patients which goes beyond merely exceeding measured values. This makes it possible to filter out emergencies quickly. Random occurrences and incorrect treatments are eliminated. A system-supported initiation of diagnosis and therapy may be performed by non-medical staff in simple treatment situations, for example, the use of a nurse instead of a physician.

It is advantageous if the medical center is broken down into several, advantageously into two, levels, a first level being provided for the routine support of the patient and a second level being provided for further support, including additional infrastructure. This contributes not only to increased efficiency but also to greater availability for the patient. Furthermore, the actual medical service providers are given the option of providing a telemedical patient service as a second level using very simple technology—ideally a PC workstation.

The first level handles all medical and/or technical inquiries of the patients. Furthermore, it is the first communication level for, in particular, physicians/nurses in private practice who provide conventional treatment for patients. The second level is initialized by the first level if consultation by a physician or specialist is necessary. It is not necessary for this service to be operated at the same location as, for example, the first level. The second level is ideally made up of a combination of a telemedical center and a conventional hospital infrastructure (hospital, physicians).

Simultaneous measurements are advantageously correlated with one another or instantaneous measurements are correlated with previous measurements for context-sensitive interpretation and evaluation of the vital data.

Advantageously, an adaptive change of a therapy plan is made as a function of the data evaluated by the telemedical center.

For the remote diagnostic monitoring and support of a patient, a device is provided including sensors and/or measuring devices for continuously recording vital data of a patient, an evaluation device for the recorded vital data with regard to their trend and the context, in particular in line with a therapy plan, a unit for preparing a record of transmission data based on the vital data for the evaluation in a medical center and a unit for signaling whether, based on the evaluation, additional vital data or information inputs of the patient are necessary, and for signaling whether the vital data are valid and have been conveyed successfully.

It is advantageous to integrate a locating unit for the patient into the device. This makes it possible to track the patient via RFID, GPS, Galileo, GSM or WLAN signals.

An acoustic signal recording in the patient's surroundings makes it possible to transfer additional information to emergency medical staff. In particular if the signal recording is automatic or may be enabled by the medical main office, it is possible to connect to the patient's home if the patient is unable to reach a telephone, etc., due to injuries or confinement to bed.

Example embodiments of the present invention will be elucidated in greater detail below with reference to the drawings.

### BRIEF DESCRIPTION OF THE DRAWINGS

FIG. 1 shows the structure of a base station and a medical center,

FIG. 2 shows the process architecture in the base station and in the medical center.

### DETAILED DESCRIPTION

The remote diagnostic monitoring and support of the patient according to example embodiments of the present invention is presented below using heart failure as an example. The use of telemedicine makes considerable benefit possible for the patient and the treating service providers, for example, physician, hospital, in the event of CHF (chronic heart failure). For the patient, these include:

independence with regard to time, for example, no waiting times due to continuous monitoring;

high gain in mobility through substitution of the follow-up visits to the physician by telemedical monitoring of the patient at home (automatic recording of vital signs and transfer to the physician);

higher reliability of care through continuous monitoring of the vital signs (threatening changes in the health condition may be detected and treated early). The patient experiences an improved quality of life;

lengthening of life, since critical health conditions, which generally result in immediate death, in particular cardiovascular, pulmonary and renal conditions, are avoided;

optimized efficacy of medication through continuous monitoring and immediate adjustment, if necessary;

reduction of the waiting time for treatment by a specialist.

The effects referred to arise as a result of process adjustments and a changed treatment structure for the patient. The primary innovations include:

the patient is monitored at home, the frequency of follow-up visits to the physician is sharply reduced, time frames of the manual process steps are automated;

the diagnosis by the physician is no longer based on a single observation (at the point in time of the visit to the physician), instead it may made more reliably based on the continuous collection/evaluation of the vital data;

the application of and compliance with therapy plans according to the most advanced state of science may be monitored and followed up centrally by specialists;

random occurrences and incorrect treatments by the family physician are eliminated (today only approximately 40% of CHF patients are treated in conformity with guidelines);

a part of the medical competence, for example, follow-up evaluation of an EKG, may be substituted by intelligent systems (pattern recognition of individual patient data, pattern recognition across all patient data). This allows non-medical staff to be employed in patient care.

It is only possible to implement the changes of the processes and structures using new technologies, both at the patient's and the physician's location (all service providers).

At the patient's location, the use of powerful measuring instruments for recording vital signs is necessary. To fulfill the increased requirement for ergonomics/operability, sensors/sensor modules are used which are directed toward miniaturization and improvement of wearing comfort, including increased measuring accuracy/precision, for example in scales, sensors for recording parameters that were previously not considered, for example, patient activity, in particular through the use of microsystem technology and communication capability, for example, via Bluetooth. Intelligent signal preparation and processing makes it possible to apply simple rules for medical interpretation of the measuring results, for example, pattern recognition for automatic diagnosis support.

The process efficiency in a medical center may be improved in the following manner:

automatic recording, processing and forwarding of the patient data (remote monitoring and data storage);

system-supported diagnosis and initiation of therapy in simple treatment situations (smart medical logic) by non-medical staff or use of a nurse instead of a physician;

centralization and bundling of activities/processes in a control center and a medical call center.

Total integration in a platform achieves the following:

integration of all systems, process steps and participants in one uniform scalable system;

smart medical logic which dynamically couples the linking of the measured values with treatment guidelines (using a feedback loop);

openness and mobility at the terminal location.

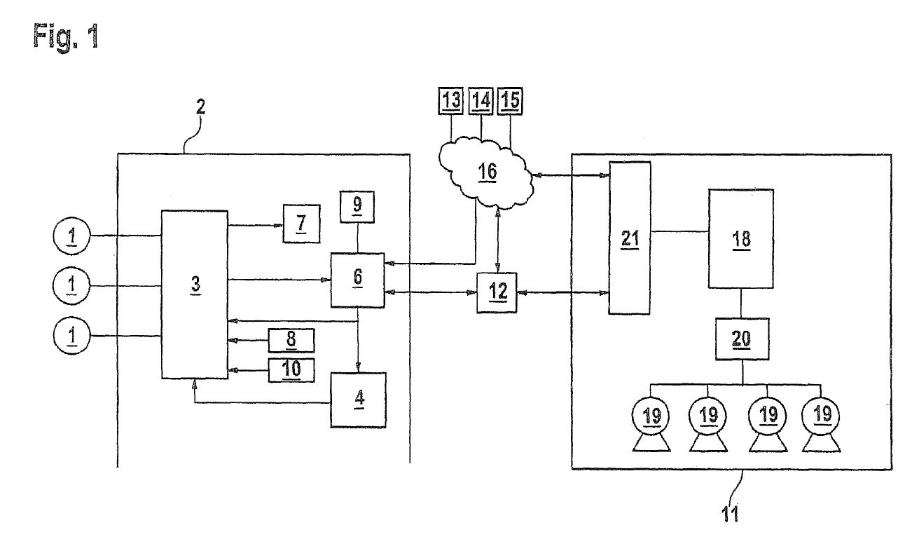

According to FIG. 1, the equipment at the patient's location is made up of sensors and/or measuring instruments 1 for recording various vital signs, a base station 2 for controlling sensors 1, signal processing of the recorded sensor/measuring signals and communication with a medical center. As FIG. 1 shows, various sensors 1 are connected to base station 2, or integrated into it, for recording a plurality of measured parameters, for example, temperature, movement, pressure, weight, blood pressure, pulse. The equipment and sensors must be appropriate to the living situation and the condition of the patient (waterproof, disinfectable, shock resistant, long-lived, insusceptible to improper use, etc.). With regard to ergonomics, the devices/sensors are configured such that they may be operated/used by laypersons, old persons, sick persons (feeble, immobile, visually impaired, etc.) and in particular by patients exhibiting low compliance (therapy acceptance and patient cooperation). It must be possible to switch off the terminals deliberately, for example, when bathing; they must have automatic/semi-automatic startup in order to avoid false alarms or non-monitoring. The devices and sensors must be small; in many cases they must be capable of being worn directly on the skin or under clothing. A maximum battery life, if possible an alternative energy supply, for example, from the patients' movement or body heat, is advantageous. A differentiation must be made between two basically different model variants,

measuring instruments which require no additional base station and thus transmit their signals directly to a telemedical center;

measuring instruments which communicate with a base station. The base station transmits the measured data to the medical center.

Both types may also be differentiated with regard to the location of the signal processing, evaluation and feedback to the measuring method:

Dumb terminal: The intelligence of the measuring and control circuit is situated in a base station or in the medical center. That is where the function of the patient terminal is controlled.

Intelligent terminal: Significant signal processing and evaluation is performed at the patient's location.

Possible feedback may be carried out immediately. Only processed data are forwarded to the medical center.

FIG. 1 shows the second variant, i.e., an intelligent terminal/base station 2. That is where the continuously recorded/measured vital data of sensors/measuring instruments 1 are interpreted and evaluated using signal technology in an evaluation device 3 with regard to their trend and the context in which they were recorded/measured. The evaluation is carried out based on a therapy plan stored in a memory 4, for example, according to the European Society of Cardiology by CHF. Based on the measured values, the therapy plan is automatically passed through a logic tree. The measured values are adapted to the therapy plan either sequentially or in parallel. Discrete values (constants, vectors, tensors) may be interpreted and compared from a precise and reproducible signal evaluation using algorithms in order to obtain information concerning the condition and trend of the patient's health condition. The necessary signal evaluations may be, for example, a filtering of the raw data via a Fourier transformation or a kernel (matrix operations) in signal patterns. For trend analyses, it is necessary to form the first derivation at the time from a regression function of chronologically successive measured values which may be obtained via iteration of polynomials. For the analysis of complex signal patterns, for example, EKG, it is possible to use self-learning algorithms, for example, non-linear mathematical methods. In the case of heart failure, body weight in particular, which was previously of little significance, is important in these patients due to the fact that fluid retention based on specific patterns in weight change may be detected, indicating a deterioration of the clinical picture. This requires appropriately sensitive scales (piezo elements) which should exceed the precision of conventional home scales by a factor of 10.

The telemedical signal processing according to example embodiments of the present invention is shown in detail in Table 1. The medical parameters (vital data) such as blood pressure, pulse activity, weight, EKG, oxygen saturation (SpO2) are measured over time t, interpreted and evaluated using signal technology, filtered in particular, Fourier-transformed, subjected to trend analysis via the first derivation of the value trend, or an analysis of a complex signal pattern is performed as well as a value assignment, for example, via self-learning iteration steps.

If necessary, additional measurements of vital data or information inputs by the patient are initiated or requested from the evaluation in line with a therapy plan. The logical decision mentioned in Table 1 is made in the medical center and is explained in connection with the description of the medical center.

Base station 2 according to FIG. 1 contains a unit 6 for preparing a record of transmission data based on the evaluated vital data for the evaluation in medical center 11. In addition, it contains a unit 7 for signaling whether, based on the evaluation, additional vital data or information inputs are necessary and for signaling whether the vital data are valid and have been transmitted successfully. In the simplest case, signaling unit 6 is made up of a display, if necessary in connection with an acoustic output, possibly a vibrating alert. This may also be used for feedback from medical center 11. An input unit 8 is provided for the input of information by the patient. Unit 6 for the preparation of transmission data is also used advantageously for the reception of information by medical center 11. The received information is on the one hand forwarded to memory 4 for possible updating of the therapy plan and on the other hand to signaling unit 6 for optical presentation on a display and/or acoustic output. Corresponding information may also be input directly into evaluation device 3, bypassing memory 4. As an alternative or in addition to input device 8, an acoustic recording device 9 is provided, in particular for the case that the patient is not able to operate input device 8. This makes it at least possible for a call for help and/or breathing sounds to be recorded. Recording device 9 may also be enabled automatically by medical center 11 and may also be coupled to a video camera in order to monitor the patient directly in the absence of inputs or in emergencies.

Base station 2 advantageously has a locating unit 10 which is also effective within buildings. In particular, combinations of various locating methods, for example, GPS, RFID, Galileo, WLAN are available for this purpose.

The data transmission from a base station 2 to a medical center 11 as well as the feedback from medical center 11 to base station 2 may be made via landline or wireless using customary methods, for example, GSM, GPRS, UMTS, ISDN, DLS, PSDN interconnected with a telecommunication provider 12. Conventional medical services such as family physician 13, emergency services 14, pharmacies 15 may be integrated into the data transfer via transmission network 16.

Since the data are confidential, it is advantageous to encrypt the data transmission between the base station and medical center 11. Such encryption is also advisable for the data transfer between sensors and/or measuring instruments 1 to base station 2.

Telemedical center 11 represents the central platform for integrating all technical functions and processes. In detail, these include:

infrastructure for data recording, data evaluation, data storage;

communication, call and data acceptance, forwarding;

control of all communication channels (speech, data, video);

control of the automatic measured value recording at the patient's location;

linking the evaluation of the measured data to a treatment plan;

providing the medical application software with patient data and therapy recommendations for a medically trained nurse;

dynamic optimization of the diagnostic and therapy plans by follow-up of therapy results;

ensuring exchange of data with other service providers in the health system, for example, physician in private practice, pharmacist, etc., possibly via an electronic patient record (e-record) or electronic medical reports.

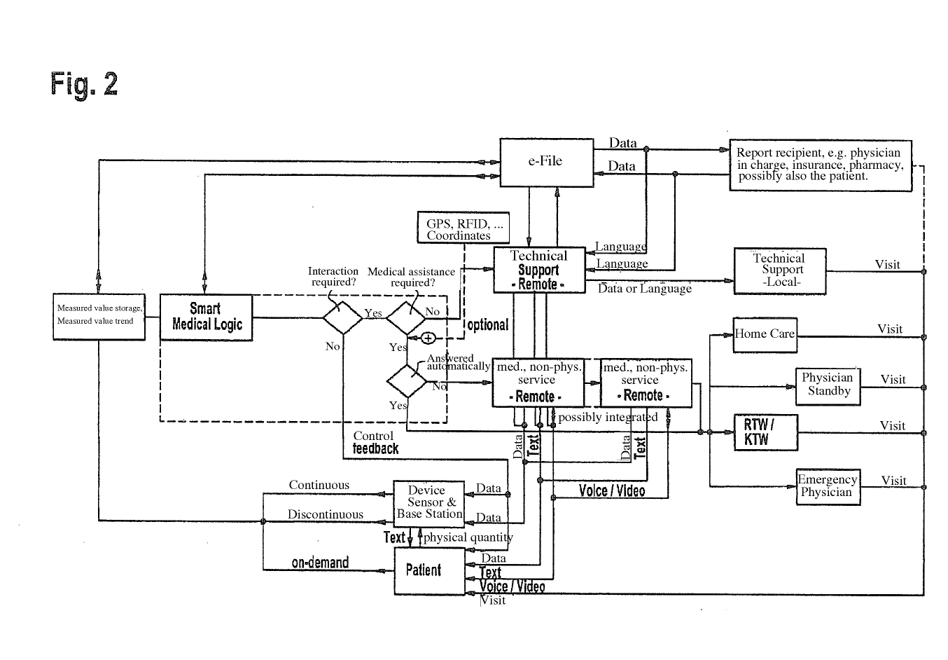

According to FIG. 2, the process in detail starts with the acquisition of patient data. A measured value recording communicates the patient's values to the smart medical logic in the medical center, based on which technical medical staff who are not physicians and physicians of the medical center are involved as needed. These three levels communicate with a technical service on site or with local medical service providers who support/treat the patient.

An aspect in the medical therapy process supported by a medical center is smart medical logic (SML). It provides:

improved diagnostic methods based on continuous measurement of various vital signs and their correlation patterns over time;

improved compliance with therapy plans. The intelligent linking of measured data with a treatment plan within the context of a decision tree may be automated and used for the support of medical care, i.e., the telemedical nurse receives a suggestion for a specific therapy instruction automatically which is necessary based on the patient history and the current measured data in connection with defined treatment plans. In a triage, the smart medical logic categorizes the patients into the status “not requiring interaction,” “requiring normal interaction” in a predefined time frame, and “immediate interaction necessary” (emergency). Based on this, the operation of an automatic control center having telemedical workstations (PC workstations) is possible in order to make optimal use of the medical center's resources;

expanded individualized therapy function: Based on the possibility of following up the efficacy of therapy plans on the patient immediately, the smart medical logic makes a learning system available. This makes it possible to adapt therapy measures individually or basically develop new therapy forms (feedback).

If base station 2 is structured simply, the previously described evaluation such as trend analyses, analysis of complex signal patterns is transferred to medical center 11. Existing medical centers usually make only one call center available which usually has only a general advising function for the patient. There is no automated integration using current measured data and analyses concerning the patient's health condition. Telemedical center 11 according to example embodiments of the present invention ensures this integration. To that end, it integrates the patient via bidirectional contact using status displays, text messages or telephone functions.

For patient care, a telemedical workstation (PC workstation) 19 in medical center 11 uses the stored data/values in the electronic patient database (electronic patient record 14). Using linking device 18, the smart medical logic makes a preselection and corresponds with a telemedical workstation 19, for example, via an http-capable Java Frontend. For performance reasons, the smart medical logic must be written in a non-object-oriented programming language. The data transfer between PC workstations 19 and linking device 18 is controlled via an application server 20. Workstation 19 establishes contact on the medical side with the conventional service providers (hospital, specialist physician, physician in private practice, emergency medical service and pharmacy). The activities involving the patient and current patient data may be inspected by the lead physician (treating specialist or family physician). The inspection is performed actively, for example, via e-medical records, e-prescriptions and also via a direct call by the medical center in, for example, an emergency. The physician may personally obtain information passively via secured and authorized access to the electronic patient record (e-record).

The transmission records, including evaluated vital data of the patients received via telecommunication device 21, are linked to a patient profile in linking device 18 using the patient data already stored in patient database 14 based on threshold values in order to detect deviations of the patient's condition from a previously established target condition based on the stored patient condition and to decide whether, based on the evaluation, an interaction with a patient is necessary immediately, in a predefined time frame or not at all. This decision is displayed in the form of feedback in base station 2 and is also reported to workstations 19 for the TM agents and is transferred to supervisors 13, 14 and 15, if necessary.

Based on the analysis of the vital data, a translation into a diagnosis and a therapy plan is also made. If necessary, the therapy plan is changed based on the current evaluation in the medical center. If the base station is an intelligent terminal, this changed therapy plan is transferred to base station 2 and filed in its memory 4 and used for the evaluation by evaluation device 3.

Medical center 11 is broken down into several levels. The first level handles all medical and/or technical inquiries of the patients. Furthermore, it is the first communication level for, in particular, physicians in private practice/nurses who provide conventional treatment for patients. The second level is initialized by the first level if consultation by a physician or specialist is necessary. It is not necessary for this service to be operated at the same location as the first level. The second level is ideally made up of a combination of a telemedical center and a conventional hospital infrastructure (hospital physicians).

FIG. 2 shows an overview of the entire process architecture including data flows.

Measured values are stored and measured value trends are formed in base station 2 which is supplied by measuring instruments and sensors. The patient supplies information for this purpose. The patient record (e-record) is supplied with these data and managed in medical center 11. This smart medical logic (SML) is primarily located in medical center 11; however, it may also be partially integrated into an intelligent base station 2. The remote medical services and technical support are located in medical center 11. Subsequent services such as local technical support, local nursing care, emergency medical service, emergency physician, are initiated by the medical center as a function of the decision (action necessary, medical assistance necessary).

1-11. (canceled)

12. A method for remote diagnostic monitoring and support of patients, comprising:
continuous recording and/or measurement of vital data of a patient;
interpreting and evaluating vital data with regard to trend and context in which they were recorded and/or measured using signal technology;
linking the vital data to a patient profile and evaluation based on threshold values in order to detect deviations of a patient's condition from a previously established target condition; and
categorizing whether, based on the evaluation, an interaction with the patient is required immediately, in a predefined time frame or not at all.

13. The method according to claim 12, wherein feedback from a medical center is sent to the patient as to whether the vital data have been transmitted successfully and are valid.

14. The method according to claim 12, wherein the vital data are evaluated in line with a therapy plan in a terminal at the patient's location and, if necessary, additional measurements of vital data or information inputs by the patient are initiated or requested from this evaluation.

15. The method according to claim 12, wherein automated decision processes are carried out in a medical center in line with therapy plans with regard to individual indications and an individual patient.

16. The method according to claim 12, wherein a medical center is broken down into several levels, a first level being provided for routine support of the patient and a second level being provided for further support, including additional infrastructure.

17. The method according to claim 12, wherein simultaneous or sequential measurements are performed for a mutual correlation for context-sensitive interpretation and evaluation of the vital data.

18. The method according to claim 14, wherein an adaptive change of a therapy plan is made as a function of the data evaluated by the telemedical center.

19. A device for remote diagnostic monitoring and support of a patient, comprising:
sensors and/or measuring instruments adapted to continuously record vital data of a patient;
an evaluation device for the recorded vital data with regard to their and context, in line with a therapy plan;
a unit adapted to prepare a record of transmission data based on the vital data for evaluation in a medical center; and
a unit adapted to signal whether, based on the evaluation, additional vital data or information inputs of the patient are necessary, and to signal whether the vital data are valid and have been transmitted successfully.

20. The device according to claim 19, wherein a locating unit is integrated into the device for the patient.

21. The device according to claim 19, wherein the device is adapted to record acoustic signals from the patient's surroundings and transmit them to a medical center.

22. A medical center for remote diagnostic monitoring and support of patients, comprising:
a telecommunication device adapted to receive and evaluate transmission records for vital data of patients and for feedback of messages to patients; and
a linking device adapted to link received vital data of a patient to a patient profile and evaluate based on threshold values in order to detect deviations of the patient's condition from a previously established target condition, and to decide whether, based on the evaluation, an interaction with a patient is necessary immediately, in a predefined time frame or not at all.
